(19) United States
(12) Patent Application Publication
(10) Pub. No.: US 2013/0247909 A1
(43) Pub. Date: Sep. 26, 2013

(54) NEBULIZER FOR USE IN MECHANICAL RESPIRATORY VENTILATION THERAPY

(71) Applicant: Mordechai Dotan, Jerusalem (IL)

(72) Inventor: Mordechai Dotan, Jerusalem (IL)

(21) Appl. No.: 13/990,075

(22) PCT Filed: Nov. 29, 2011
(86) PCT No.: PCT/IL2011/000910

§ 371 Date: May 29, 2013

Related U.S. Application Data
(60) Provisional application No. 61/417,902, filed on Nov. 30, 2010.

(51) Int. Cl. A61M 16/14; A61M 16/08; A61M 16/20; A61M 16/00

(52) U.S. Cl. CPC A61M 16/14; A61M 16/0057; A61M 16/0816; A61M 16/208; A61M 16/0875
USPC 128/203.12

(57) ABSTRACT

A nebulizer for use in mechanical respiratory therapy that allows for the introduction of medicaments in the form of aerosols into the system without causing interruption to the therapy being provided to the patient.

### FIELD OF THE INVENTION

The present invention relates generally to the field of respiratory ventilation therapy, and more particularly to a nebulizer for delivery of medications in the form of aerosols to a patient attached to a mechanical ventilator and receiving respiratory ventilation therapy.

### BACKGROUND OF THE INVENTION

Respiratory ventilation therapy refers to forms of treatment used to assist breathing function in patients with a variety of diseases, conditions, or injuries. Typically, respiratory ventilation is provided for patients suffering from acute or chronic respiratory failure. In general, the treatment involves attaching the patient to a ventilator which is a mechanical and/or electrical device that is adapted to enable the respiratory processes to continue by providing a flow of respiratory gasses at a rate, volume and pressure needed by the patient. The patient typically is connected to the ventilator via an endotracheal tube inserted either through his nose or mouth, or through a surgical opening made in his trachea.

Patients for whom mechanical respiratory ventilation is necessary typically also need to be treated with one or more medicaments that for optimal effectiveness should also be delivered directly to their lungs, rather than administered parenterally or otherwise. Typically, such medicaments are provided by the drug manufacturer in liquid form, and these liquids must first be converted into an aerosol of tiny droplets before they can be administered to the lungs of the patient. The conversion of the liquid medicament into an aerosol typically is effected by means of a device called a nebulizer that is incorporated into the respiratory ventilation apparatus. The nebulizer is a container into which a measured amount of the liquid medicament is inserted. Oxygen (or a mixture of respiratory gasses) under pressure is directed into the container, the liquid in the container is aerosolized, and the aerosol is then mixed with the respiratory gasses flowing from the ventilator to the patient thereby reaching the lungs of the patient.

Once a patient is attached to a mechanical ventilator, all of his respiratory functions are provided by the ventilator—including the flow to his lungs of a volume of a mixture of gasses at a rate necessary for respiration, the application of sufficient pressure to inflate the lungs so as to enable inhalation, and cyclically allowing for the relaxation/deflation of the lungs so as to enable exhalation of carbon dioxide and other bi-products of respiration. All of these functions must occur in a regular, timed sequence, and must be maintained without interruption; otherwise the patient's lungs may collapse and he may cease to breathe.

Surprisingly, however, the nebulizers typically in use in hospitals today for delivery of medicaments to patients attached to mechanical ventilators are constructed in a manner such that their use often endangers the patients by undermining the integrity of the system. These nebulizers typically are constructed of a first portion that serves as a reservoir for receiving a measured amount of the medicament and in which the medicament is aerosolized (when oxygen or an oxygen/gas mixture flows thereto), and a second portion comprising a number of ports adapted for connecting various tubes thereto, including an entry port for conveying oxygen under pressure to the nebulizer, and an exit port for directing the aerosolized medicament to the tube conveying the respiratory gasses to the patient. Typically, the first and second portions of these nebulizers are manufactured as distinct sections that are adapted to fit one into the other, such as via a simple screw-type mechanism, after the insertion of the medicament. Because of this configuration, each and every time that it becomes necessary to fill or refill the reservoir with the liquid medication—and this typically occurs between 2 and 12 times a day for patients on a ventilator—the two portions of the nebulizer must be separated one from another. But when this occurs, the result is potentially catastrophic for the patient. There is an immediate loss of the volume of gasses needed by the patient for respiration, since all (or most) of the gasses that continue to flow from the ventilator now escape to the ambient environment. In addition, there is an immediate loss of pressure in the system, such that the pressure necessary for the patient to inhale (continue breathing) falls suddenly. The result is great distress for the patient—he feels as if he is choking—and since there is insufficient pressure to force the patient's lungs to expand, the lungs may collapse causing the alveoli of the lungs to adhere to each other (often an irreversible process). In addition, since the system of tubing is no longer closed, but is open to the ambient environment, the patient is exposed to an enhanced risk of infection from bacteria and other pathogens in the ambient environment. At the same time, the medical staff attending to the patient, and anyone else in the vicinity (other patients, family or visitors) are exposed to pathogens from the patient that are now being circulated from the open system to the ambient environment.

Thus, the nebulizers in use today are woefully inadequate. Clearly there is a need for an improved nebulizer for use in ventilation therapy that overcomes these drawbacks and provides a safe and sterile means for introducing medication into the nebulizer without undermining the integrity of the system and endangering the lives of the patients.

### SUMMARY OF THE INVENTION

The present invention provides an improved nebulizer for delivering medication in the form of an aerosol to a patient attached to a mechanical ventilator and receiving respiratory ventilation therapy, the improvement comprising means for inserting the medication into the nebulizer without causing any disruption to the respiratory ventilation process.

In accordance with a preferred embodiment, the nebulizer of the present invention consists of a container having a volume sufficient to hold a therapeutically effective amount of a liquid medicament and to enable aerosolizing therein of the medicament upon the inflow thereto of a respiratory gas, the container comprising a first port adapted for attachment to a source of a respiratory gas, a second port adapted for attachment to the apparatus providing respiratory ventilation to the patient, and a third port adapted for inserting the medicament into the container without causing interruption to the respiratory ventilation therapy being provided to the patient.

In accordance with a preferred embodiment, the third port comprises a one-way valve adapted to allow insertion of medication into the nebulizer without causing a loss of pressure or gas volume, or a reduction in the flow rate of the oxygen/gas mixture, and without allowing the venting to the ambient environment of respiratory gasses being provided to the patient.

In accordance with a preferred embodiment, the third port comprises an insertion point for insertion of the medicament via a needle of a syringe, or any other apparatus capable of delivery of the medicament through the insertion point.

In a preferred embodiment, the insertion point is sealed with an elastomeric material.

In accordance with a preferred embodiment of the invention, the container has a unitary structure. The container may be constructed of any material suitable for use in a hospital environment, such as a hard plastic material.

Alternatively, the container may be constructed of two or more pieces that are conjoined prior to attachment to the respiratory ventilation apparatus. For maximum utility and benefit, the pieces are conjoined in a manner preventing their separation after the container is attached to the respiratory ventilation apparatus.

In accordance with yet another embodiment, the container further comprises a configuration adapted to facilitate aerosolizing of the medicament. In one aspect, the configuration comprises a bottom portion that tapers downward; in another aspect, the configuration comprises a narrow tube connected to the first port positioned so as to direct the oxygen (or mixture of respiratory gasses) flowing into the container towards the bottom of the container.

In accordance with yet another embodiment, the container further comprises a fourth port and apparatus adapted for draining residual liquid from the container without causing interruption to the respiratory ventilation therapy being provided to the patient. The apparatus comprises a collector and a valve mechanism having at least two positions, one position enabling drainage of the residual liquid from the nebulizer into the collector, and a second position enabling drainage of liquid from the collector.

### BRIEF DESCRIPTION OF THE DRAWINGS

The features of the present invention will be better understood with reference to the attached drawings wherein:

FIG. 1 presents a schematic view of a typical mechanical respiratory ventilation system that incorporates a nebulizer for dispensing medication to a patient; and

FIG. 2 presents a schematic view of the structure of one preferred embodiment of a nebulizer in accordance with the present invention.

FIG. 3 presents a schematic view of the structure of another preferred embodiment of a nebulizer in accordance with the present invention.

### DETAILED DESCRIPTION OF THE INVENTION

The present invention provides an improved nebulizer adapted to be used in connection with mechanical respiratory ventilation systems, such as those used in hospitals, that allows for the introduction of medicaments in the form of aerosols into the system without violating the integrity of the closed system, thereby preventing the deleterious effects experienced by patients today who are attached to ventilators when the nebulizers are opened to insert a medication.

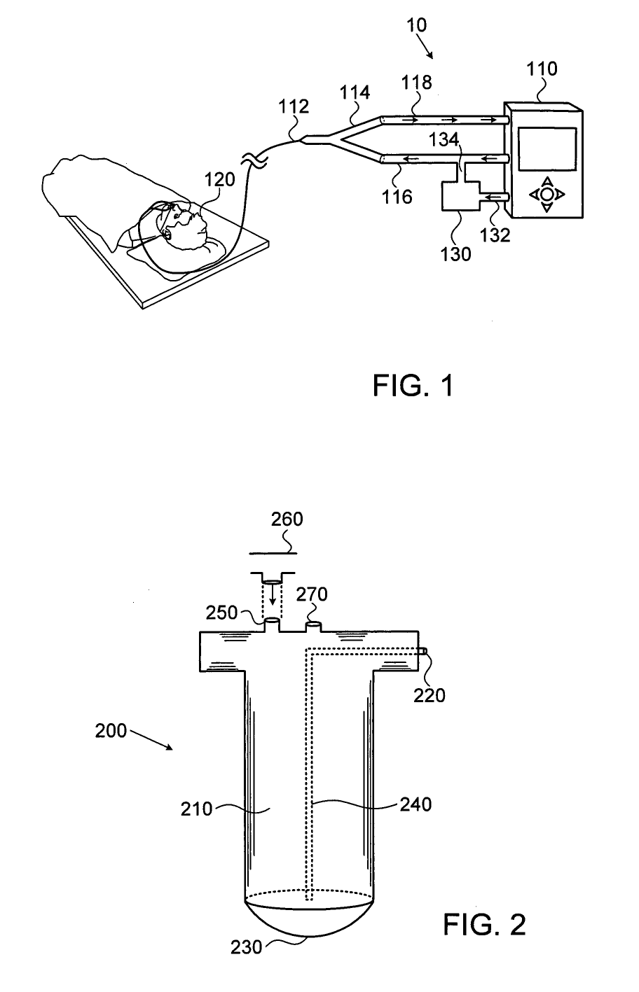

The basic apparatus 10 of a typical respiratory ventilation system, in connection with which the present invention may be used, is represented graphically in FIG. 1. Apparatus 10 comprises a mechanical ventilator 110, for providing air and/or oxygen and/or other respiratory gasses (e.g., NO) to a patient 120. Ventilator 110 has one or more entry ports (not shown) adapted for attachment to sources of the gasses needed in connection with the mechanical ventilation of patients. Prior to the patient's attachment to the apparatus, an endotracheal tube 112 is inserted into the patient either through his nose or mouth, or through a surgical opening made in his trachea. Tube 112 is then attached to the ventilator apparatus typically via a Y-type connector 114 which in turn is connected to conduits 116 and 118. Conduit 116 is adapted to receive one or more of the gasses under controlled pressure from ventilator 110 and direct them to the patient; conduit 118 is adapted to receive carbon dioxide and other bi-products of exhalation from the patient and direct them back to the ventilator. Conduits 116 and 118 may consist of a series of conduits, connectors, filters and the like (all not shown).

Ventilator 110 is powered by a power source (not shown), such as electricity or compressed gas. Associated with ventilator 110, or built-in with it, are control and monitoring systems (not shown) that enable a health professional to determine various parameters of the system, such as the volume of the gas, the flow pressure, temperature, the timing of inhalation and exhalation, and the like.

Typically, the oxygen and other gasses delivered to the patient must be both warmed and humidified in order to prevent drying out of lung tissue. To that end, a humidifier (not shown) is hooked into the circuit of tubes at a convenient place, typically in proximity to ventilator 110.

In order to deliver medication to the lungs of the patient simultaneously with the delivery of the respiratory gasses, a nebulizer 130 is provided in a manner that enables it to be removeably attachable to the system. Typically, nebulizer 130 is attached via a conduit 132 to a source of oxygen (or a mixture of oxygen and other respiratory gasses) under pressure; in the diagram, the source of oxygen is ventilator 110, but a source independent of the ventilator will work equally as well. An exit port 134 of the nebulizer is connected to conduit 116. When the nebulizer is filled with a liquid medicament, a flow of oxygen (or a mixture of oxygen and other respiratory gasses) is directed into the nebulizer which acts to convert the liquid to an aerosol; the aerosol is then directed to the patient via conduit 116.

Once the patient is attached to ventilator 110, his normal respiratory functions are provided by the ventilator—including the flow to his lungs of a volume of air and other gasses necessary for inhalation, the application of sufficient pressure to inflate the lungs so as to enable inhalation, and cyclically allowing for the relaxation/deflation of the lungs so as to enable exhalation of carbon dioxide and other bi-products of respiration. All of these functions must occur in a regular, timed sequence, and must be maintained without interruption; otherwise the patient may cease to breathe.

Surprisingly, however, the nebulizers typically in use in hospitals today for delivery of medicaments to patients attached to ventilators are constructed in a manner such that their use often undermines the integrity of the system and may even endanger the lives of the patients. These nebulizers typically are constructed of a first portion that serves as a reservoir for receiving a measured amount of the medicament and in which the medicament is aerosolized (when oxygen flows thereto), and a second portion comprising a number of ports adapted for connecting various tubes thereto, including an entry port for conveying oxygen under pressure to the nebulizer, and an exit port for directing the aerosolized medicament to the tube conveying the respiratory gasses to the patient. Typically, the first and second portions of these nebulizers are manufactured as distinct sections that are adapted to fit one into the other, such as via a simple screw mechanism, after the insertion of the medicament. Because of this configuration, each and every time that it becomes necessary to fill or refill the reservoir with the liquid medication—and this typically occurs between 2 and 12 times a day for patients on a ventilator—the two portions of the nebulizer must be separated one from another. But when this occurs, the result is potentially catastrophic for the patient. There is an immediate loss of the volume of gasses needed by the patient for respiration, since all (or most) of the gasses that continue to flow from the ventilator now escape to the ambient environment. In addition, there is an immediate loss of pressure in the system, such that the pressure necessary for the patient to inhale (continue breathing) falls suddenly. The result is great distress for the patient —he feels as if he is choking—and since there is insufficient pressure to force the patient's lungs to expand, the lungs may collapse because the alveoli of the lungs adhere to each other (often an irreversible process). In addition, since the system of tubing is no longer closed, but is open to the ambient environment, the patient is exposed to an enhanced risk of infection from bacteria and other pathogens in the ambient environment. At the same time, the medical staff attending to the patient and anyone else in the vicinity (other patients, family or visitors) are also at risk of exposure to pathogens originating from the patient that are now being circulated from the open system to the ambient environment.

The nebulizer of the present invention provides a simple and effective way for overcoming these drawbacks. It allows a patient attached to a ventilator to receive repeated dosages of aerosolized medication without exposing him to the dangers described above inherent in the nebulizers commonly in use today.

In accordance with a preferred embodiment (FIG. 2), in its simplest form, the nebulizer of the present invention consists of a container 200, having an inner volume 210 that will accommodate a therapeutically effective amount of a liquid medication and yet have sufficient additional space to allow for the aerosolizing of the liquid upon the introduction of a flow of oxygen (or a mixture of respiratory gasses) thereto. In one embodiment, the container may accommodate up to about 20 cc of a liquid medication; however, containers both larger and smaller than this are also within the scope of the present invention.

Container 200 may be formed of any suitable material. Typically, it is formed of a rigid plastic as is known in the art and as is commonly used for many short-term-use or disposable medical devices.

In a preferred embodiment, container 200 has a unitary structure and comprises three ports as more fully described below. In other embodiments, container 200 may be constructed of two or more pieces that are conjoined prior to attachment to the respiratory ventilation apparatus. However, for maximum utility and benefit, the pieces are conjoined in a manner preventing their separation after the container is attached to the respiratory ventilation apparatus.

Container 200 has an entry port 220 adapted for connection to a conduit (such as conduit 132 in FIG. 1) delivering respiratory gasses under pressure. Container 200 preferably has a shape and internal structure that facilitates the process of aerosolizing the liquid medication. In one embodiment shown in FIG. 2, the bottom of container 200 is tapered to a point 230, and the respiratory gasses flowing into the container via entry port 220 are directed towards the bottom of the container by a narrow conduit 240 which extends almost to the bottom of the container. However, these features are merely representative of the type of features that may be incorporated into the structure of the nebulizer of the present invention to accomplish this purpose, and that could be readily be designed by a person of the art.

Container 200 has an exit port 250 adapted for connection to the conduit delivering the respiratory gasses to the patient (such as conduit 116 in FIG. 1); typically, the connection is effected via a “T”-shaped connector 260 as shown, but other forms of connectors are also possible within the scope of the present invention. Once container 200 is attached both to the source of oxygen via entry port 220 and to the conduit delivering the respiratory gasses to the patient via exit port 250, container 200 is effectively sealed to the ambient environment, and forms an integral component of the closed circuit that is providing the required sequenced volume of respiratory gasses under pressure to the patient.

Container 200 has one additional port 270 adapted for enabling the introduction of liquid medication into the container. However, port 270 is a sealed port—sealed by any means adapted both to allow the introduction of a liquid medicament into the container and yet to prevent the escape of aerosol or respiratory gasses from the container. In one embodiment, the port is sealed by a one-way valve, that allows for the insertion of a medicament into the container but does not allow for the exit of any liquid or gas from the container. In a preferred embodiment, port 270 is sealed by an “insertion point,” i.e., by an elastomeric material, as is known in the art, such as that found on devices designed for the introduction of fluids into intravenous feeds. The elastomeric material of the insertion point may be penetrated by the needle of a syringe without destroying the utility of the material as a seal. In the practice of the present invention, a standard syringe is filled with a measured amount of the medication needed to treat the patient attached to the ventilator, and the liquid is inserted into the nebulizer (container 200) by piercing the covering of port 270 with the needle of the syringe. Once all the liquid in the syringe is emptied into container 200, the needle is withdrawn, without damaging the seal, and without causing any loss in the volume of the gasses being delivered to the patient or without any loss of pressure needed to maintain the regular cycle of respiration for the patient. It will also be appreciated that as soon as the liquid medication is introduced into the container via the insertion point, (oxygen-respiratory gasses) under pressure may be directed to the nebulizer to start the process of aerosolizing the liquid, and the aerosol can then begin to flow towards the patient via exit port 250.

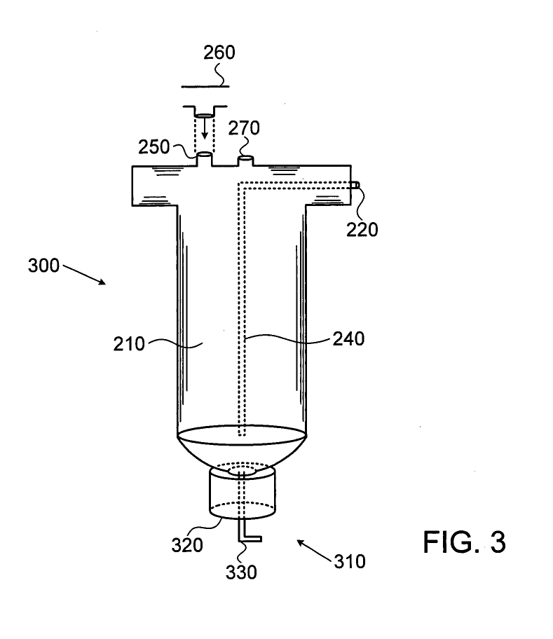

Another preferred embodiment of the nebulizer of the present invention may be appreciated with reference to FIG. 3. As can be seen in FIG. 3, the nebulizer 300 of this embodiment is identical in most aspects to nebulizer 200 described above with reference to FIG. 2. However, nebulizer 300 has an additional feature—an apparatus and mechanism 310 either integral to the nebulizer or removeably attachable to the nebulizer at its bottom portion that is adapted to enable effective removal of residual and/or unwanted medicament from the nebulizer without interruption to the ongoing operation of the respiratory ventilation system. In its simplest form, apparatus and mechanism 310 comprises a container 320 and a valve 330. Valve 330 may take the form of a stopcock or any similar device that may be operated in at least two positions In one position, the valve is operable to open an exit port (not shown) on the bottom of the nebulizer to allow for the residual medicament to drip into the collector. When the valve is in this position, the integrity of the nebulizer is maintained, since it remains sealed to the ambient environment. In a second position, the valve is operable to seal the exit port on the bottom of the nebulizer while opening up a drainage port (not shown) on the bottom of the collector. When the valve is in this position, any liquid that had dripped into the collector may be drained out and yet the integrity of the nebulizer and the respiratory system is still maintained

It will be appreciated that the embodiments of the present invention described above are only provided for illustrative purposes only. A person of the art will appreciate that many other structural variations are possible within the scope of the present invention which is limited only by the following claims:

1. A nebulizer for use in conjunction with apparatus providing mechanical respiratory ventilation therapy to a patient, the nebulizer comprising:
a container having a volume sufficient to hold a therapeutically effective amount of a liquid medicament and to enable aerosolizing therein of the medicament upon the inflow thereto of a respiratory gas, the container comprising:
a first port adapted for attachment to a source of a respiratory gas,
a second port adapted for attachment to the apparatus providing respiratory ventilation to the patient,
a third port adapted for inserting the medicament into the container without causing interruption to the respiratory ventilation therapy being provided to the patient; and
a fourth port adapted for draining residual liquid from said nebulizer without causing interruption to the respiratory ventilation therapy being provided to the patient.

2. The nebulizer according to claim 1 wherein the container is of a unitary structure.

3. The nebulizer according to claim 1 wherein the third port is a one-way valve allowing introduction of the medicament without allowing outflow of any respiratory gas or aerosol.

4. The nebulizer according to claim 1 wherein the third port comprises a sealed insertion point for insertion of a needle of a syringe.

5. The nebulizer according to claim 4 wherein the insertion point is sealed with an elastomeric material.

6. The nebulizer according to claim 1 wherein the container comprises an internal configuration that is adapted to facilitate the aerosolizing of the medicament therein.

7. The nebulizer according to claim 6 wherein the configuration comprises a bottom portion that is tapered downward.

8. The nebulizer according to claim 6 wherein the configuration comprises a narrow tube extending from the first port and directing the inflow of respiratory gas towards the bottom of the container.

9. The nebulizer according to claim 1 wherein the container is of sufficient size to hold up to about 20 cc of the medicament.

10. (canceled)

11. The nebulizer according to claim 1 wherein the fourth port comprises a collecting portion and a one way valve, wherein in a first position, the one way valve opens an exit port on the bottom of the nebulizer to allow for the residual medicament or fluids to drip into the collecting portion so that the integrity of the nebulizer is maintained, wherein in a second position, the one way valve seals the exit port on the bottom of the nebulizer while opening up a drainage port on the bottom of the collecting portion wherein the liquid in the collecting portion may be drained out and yet the integrity of the nebulizer.
